(19) United States
(12) Patent Application Publication
(10) Pub. No.: US 2025/0160929 A1
(43) Pub. Date: May 22, 2025

(54) SITUATIONAL CONTROL OF SMART SURGICAL DEVICES

(71) Applicant: Cilag GmbH International, Zug (CH)

(72) Inventors: Frederick E. Shelton, IV, Hillsboro, OH (US); Jason L. Harris, Lebanon, OH (US); Jacqueline Corrigan Aronhalt, Loveland, OH (US)

(21) Appl. No.: 18/810,175

(22) Filed: Aug. 20, 2024

Related U.S. Application Data
(60) Provisional application No. 63/602,040, filed on Nov. 22, 2023.
(60) Provisional application No. 63/601,998, filed on Nov. 22, 2023.
(60) Provisional application No. 63/602,998, filed on Nov. 27, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 18/14; A61B 17/00; A61B 17/072; A61B 17/32; A61B 18/00; A61B 90/00

(52) U.S. Cl. CPC A61B 18/1445; A61B 17/07207; A61B 17/320092; A61B 90/361; A61B 90/37; A61B 2017/00022; A61B 2017/00061; A61B 2017/00221; A61B 2017/00225; A61B 2017/32007; A61B 2017/320093; A61B 2017/320095; A61B 2018/0063; A61B 2018/00642; A61B 2018/00702; A61B 2018/00773; A61B 2018/00904; A61B 2018/1452; A61B 2090/373; A61B 2217/007; A61B 2218/002; A61B 2218/007; A61B 2218/008

(57) ABSTRACT

In examples, a first surgical device may be configured to detect a user operation at the first surgical device. The first surgical device may obtain data associated with the user operation. The first surgical device may determine, based on the data, whether the user operation is associated with a second surgical device separate from the first surgical device. The first surgical device may generate a control signal for controlling the second surgical device based on the determination that the user operation is associated with the second surgical device.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices.

### SUMMARY

Examples herein provide the ability for the surgeon to control another surgical device from the surgical device is surgeon is currently using and/or holding, potentially preventing the need for the surgeon to put down the surgical device and leave the patient.

In examples, a first surgical device may detect a user operating at the first surgical device. The first surgical device may obtain data related to a device operating condition associated with the user operation. In examples, the data related to the device operating condition may be related to a device location, a device orientation, or a device directionality relative to a surgical environment. The first surgical device may determine, based on the data related to the device operating condition, whether the user operation is associated with the first surgical device or another device (e.g., a second surgical device).

In examples, the data related to the device operating condition may indicate that the user operation corresponds to an invalid input for the first surgical device. Based on the determination that the data related to the device operating condition indicates that the user operation corresponds to an invalid input for operating the first surgical device, the first surgical device may determine that the user operation is associated with operating the second surgical device separate from the first surgical device. Based on the determination that the user operation is associated with the second surgical device, the first surgical device may generate a control signal for controlling the second surgical device.

In examples, the first surgical device may be a handheld surgical instrument having an end-effector and the second surgical may be an advanced imaging device, a smoke evacuator, an irrigation pump, etc. In examples, the first surgical may determine that the user operation corresponds to an invalid input for operating the first surgical device based on the data related to the device operating condition indicating that the handheld surgical instrument is operating with the end-effector outside a proximity of the tissue when the user operation is performed. Based on the determination that the data related to the device operating condition indicates that the user operation corresponds to an invalid input for operating the first surgical device, the first surgical device may determine that the user operation is used to reposition a focal center of an image of the advanced imaging device, to change an imaging of the advanced imaging device among fluorescing imaging, multi-spectral imaging, and visible light imaging. In some examples, based on the determination that the data related to the device operating condition indicates that the user operation corresponds to an invalid input for operating the first surgical device, the first surgical device may determine that the user operation is used to operate a smoke evacuator and/or an irrigation pump.

In examples, the data related to the device operating condition associated with the user operation may be shown via a surgical field of view. The surgical field of view may be configured to display the second surgical device to control. Based on the surgical field of view showing that the surgical device is operating with an end effector outside a proximity of a tissue when the user operation is performed, the first surgical device may determine that the user operation corresponds to an invalid input for operating the first surgical device. Based on the determination that the user operation corresponds to an invalid input for operating the first surgical device, the device position (e.g., the position of the effector of the surgical instrument) within the surgical field of view may be used to select the second surgical device to control via the user operation. In some examples, different positions of the end effector may correspond to different operations and/or control signals at the second surgical device.

In examples, a surgical computing system (e.g., surgical hub) may be configured to control the second surgical device. The surgical computing system may determine whether a user operation corresponds to an invalid input for operating the first surgical device and may control the second surgical device based on the determination.

### BRIEF DESCRIPTION OF THE DRAWINGS

FIG. 1 illustrates a block diagram of a computer-implemented surgical system.

FIG. 2 illustrates an example surgical system in a surgical operating room.

FIG. 3 illustrates an example surgical hub paired with various systems.

FIG. 4 illustrates an example of a situationally aware surgical system.

FIG. 5 illustrates an example surgical system that may include a surgical instrument.

FIG. 6 illustrates an example of a surgical system including a first surgical device controlling the operation of a second surgical device.

FIG. 7 illustrates a communication interface providing the ability for the first surgical device to control the second surgical device.

FIG. 8 illustrates an example flow diagram of a process for a first surgical device controlling the operation of a second surgical device.

### DETAILED DESCRIPTION

Surgical procedures may involve many surgical devices within a surgical system. Surgeons and HCPs may be responsible for operating many of these surgical devices during surgical procedures. Operating these devices in the often fast paced and high-pressure environment of performing surgical procedures may be difficult. For example, a surgeon may be holding one surgical instrument and may need to access other surgical instruments at or near the same time. Putting one surgical instrument down to operate another surgical instrument is inefficient and may lead to valuable time being lost when performing surgical procedures. Examples herein provide more efficient ways of operating multiple surgical instruments within a surgical system.

Surgical instruments may be configured to situationally control other surgical instruments. In examples, a surgical device may be configured to communicate with another surgical device in a surgical network. A user may perform a user operation associated with the surgical device (e.g., firing an end effector of a surgical device). Depending on data related to the device condition of the surgical device (e.g., the location or orientation of the end-effector), the surgical device may determine the surgical device is meant to control another surgical device instead of itself. In examples, the end effector of a surgical device may not be proximate to a tissue when the user fires the surgical device. Based on the end effector not being proximate to the issue, instead of firing the surgical device for cutting tissue, the user operation of the surgical device (e.g., the trigger(s), button(s), knob(s) of the surgical device) may be used to control another device such as an advanced imaging device.

Examples herein may allow surgical devices to interpret whether a user operation is intended to control itself or is intended to control other surgical devices based on data obtained (e.g., operating conditions). The ability of surgical devices to control other surgical devices within the surgical network may prevent surgeons and HCPs from putting down one surgical device to operate another surgical device. This may save valuable time during surgical procedures and may lead to better and more efficient surgical outcomes.

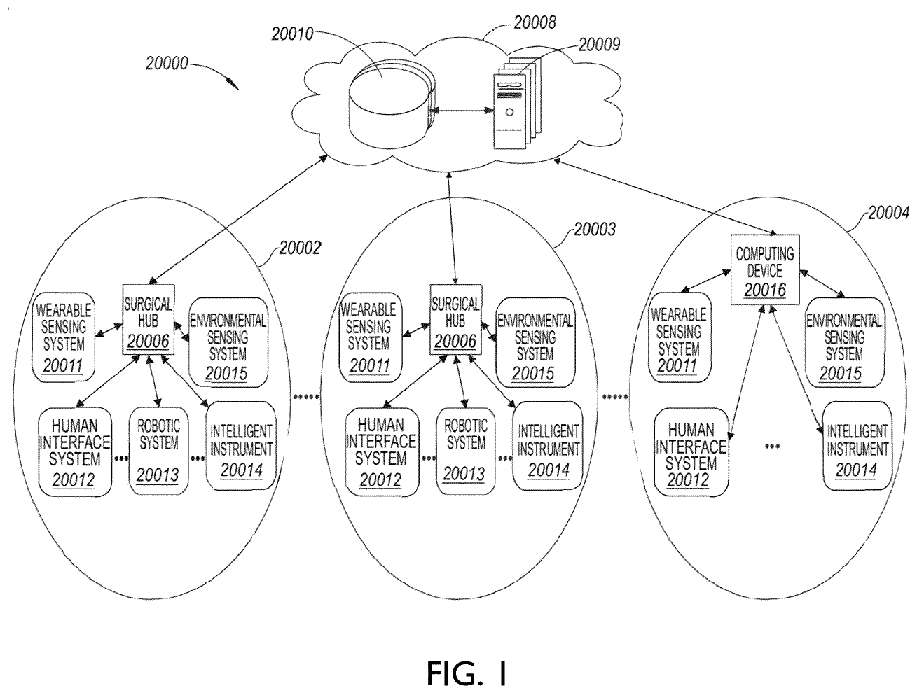

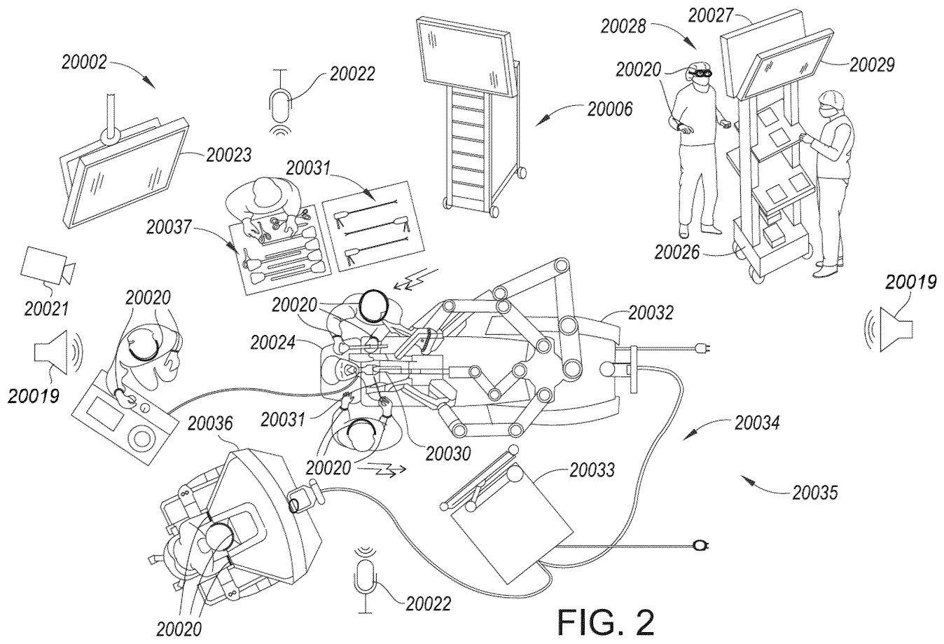

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, encephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

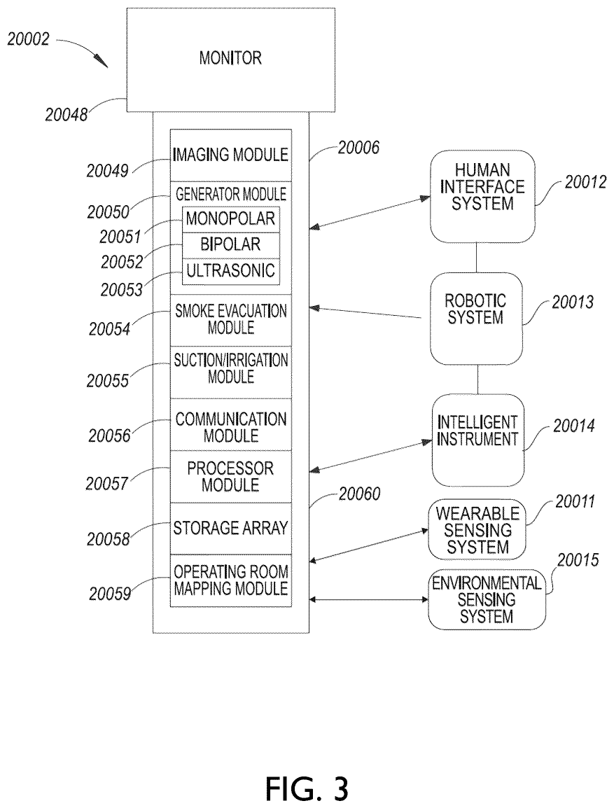

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

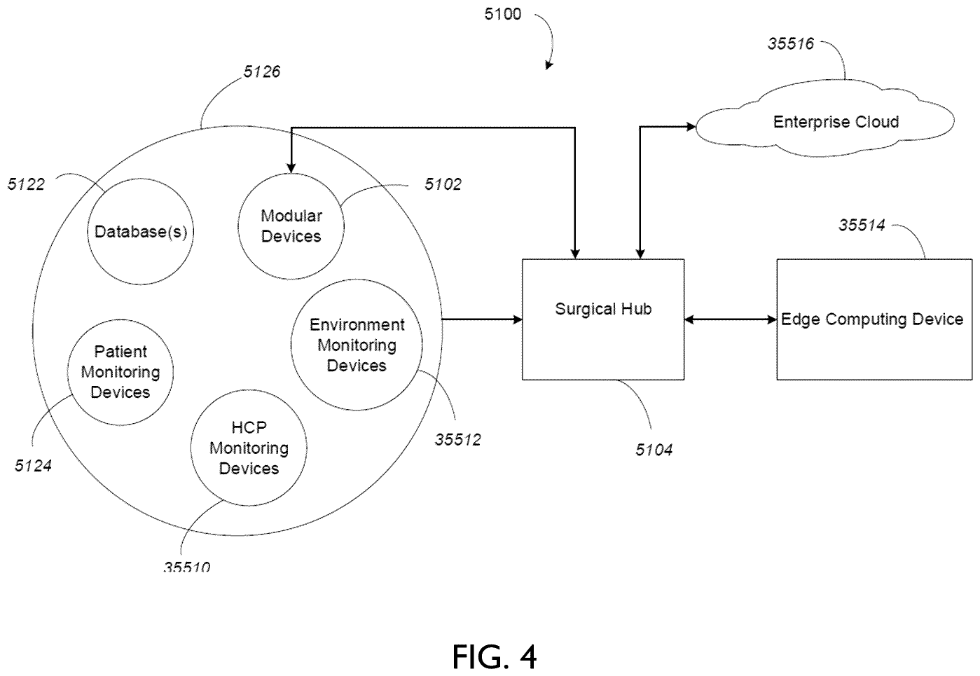

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

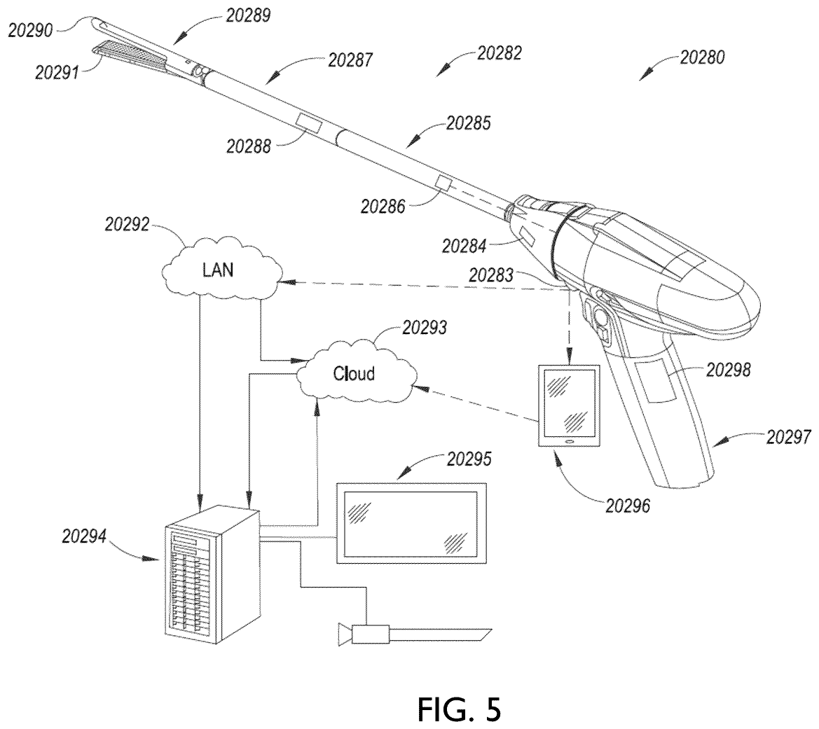

FIG. 5 illustrates an example surgical system 20280 that may include a surgical instrument 20282. The surgical instrument 20282 may correspond with the first surgical device described below. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

In fast paced surgical environments, surgeons and HCPs may be responsible for many surgical devices at a time when performing surgeries. This may require leaving or putting down one surgical device while performing surgery on a patient to operate another surgical device, potentially taking up important time and leading to worse surgical outcomes. Examples herein provide the ability for the surgeon to control another surgical device in the operating room from the surgical device is surgeon is currently using and/or holding, potentially preventing the need for the surgeon to put down the surgical device and leave the patient.

A surgical device may be meant to perform a specific surgical task. For example, a surgical device (e.g., a surgical energy device (e.g., RF, ultrasonic, or combo energy device) or surgical stapler) may be meant to use an end effector to cut tissue. In examples herein, if the surgical device is not performing the specific surgical task, it may instead control another surgical device in the operating room. For example, if the end effector of the surgical device is not proximate to a tissue to be cut, it may be used to control an advanced imaging device using the mechanisms it normally uses for cutting the tissue. For example, if the user pulls a trigger normally used to cut the tissue, it may instead be used to zoom in on an advanced imaging device or to see a different view on the advanced imaging device. This may prevent the surgeon from needing to put down the surgical device in order to adjust the view of the advanced imaging device, potentially saving important time and leading to improved surgical outcomes.

In examples, a first surgical device may detect a user operating at the first surgical device. The first surgical device may obtain data related to a device operating condition associated with the user operation. In examples, the data related to the device operating condition is related to a device location, a device orientation, or a device directionality relative to a surgical environment. The first surgical device may determine, based on the data related to the device operating condition, whether the user operation is associated with the first surgical device or the second surgical device.

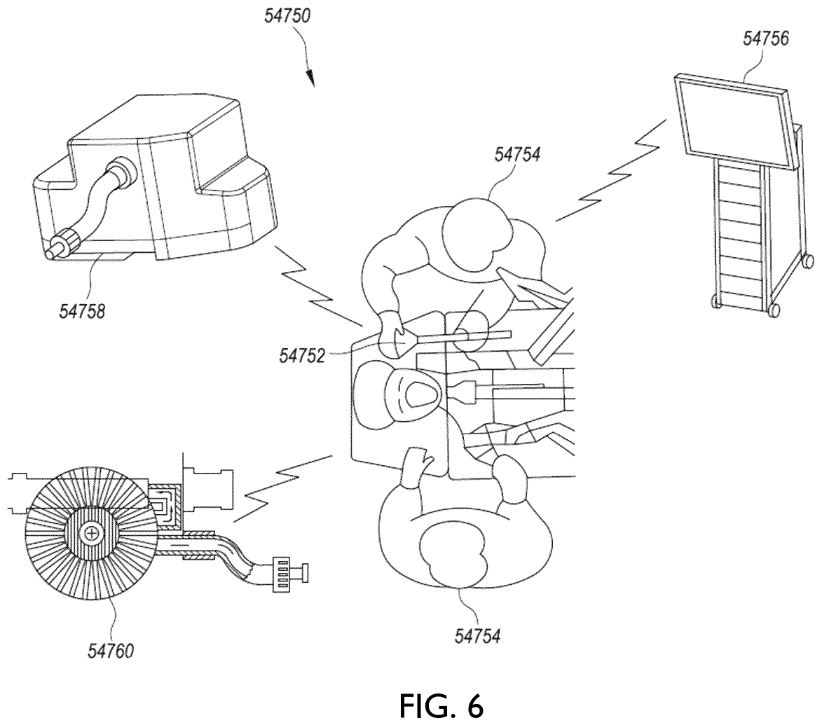

FIG. 6 illustrates an example of a surgical system that includes a first surgical device controlling the operation of a second surgical device. The first surgical device 54752 may detect a user operation performed by a user 54754 (e.g., surgeon(s), HCP(s), etc.) associated with the first surgical device 54752.

In examples, the first surgical device 54752 may be a handheld advanced energy device for adaptive control of any combination of monopolar RF, bipolar RF, or ultrasonic tissue welding. In examples, the first surgical device 54752 may communicate with a surgical hub (not shown) via wireless communications or directly through a wired connection. In examples, the first surgical device 54752 may be connected to an energy generator, which may be connected to the surgical hub. As such, the surgical hub may receive control signals from the first surgical device 54752 and pass it to a second surgical device, such as an advanced imaging device 54756, a smoke evacuator 54758, an irrigation system 54760, generator(s) (not shown), an insufflation system (not shown), a cooling system (not shown), etc. In examples, the first surgical device 54752 may communicate via BlueTooth with the second surgical device.

In examples, the first surgical device 54752 may be a handheld powered stapler. The handheld powered stapler may be configured with a powered movement. The handheld powered stapler may be configured with placement (e.g., articulation) aspects. The handled powered stapler may be configured to communicate (e.g., via BlueTooth) with other surgical devices (e.g., the second surgical device). In examples, the first surgical device 54752 may be a monopolar RF cautery pencil.

The user operation performed on the first surgical device 54752 may be pulling a trigger on the handheld advanced energy device. Pulling the trigger may squeeze the jaw members of the end-effector of the handheld advanced energy device to grasp tissue. In examples, the trigger may be a one stage trigger or may be a two stage trigger. For the one stage trigger, the trigger may be squeezed to close the jaw members. Once the jaw members are closed, the RF generator may be energized to seal the tissue. For the two stage trigger, the trigger may be squeezed part of the way to close the jaw members. The trigger may (e.g., may then) be further squeezed the rest of the way to energize the RF generator to seal the tissue.

In examples, the user operation performed on the first surgical device 54752 may be to press a firing button on the handheld powered surgical device. Pressing the firing button on the handheld powered surgical device may squeeze the jaw members of the end-effector. In examples, the user operation performed on the first surgical device 54752 may be to turn a rotation knob coupled to the shaft assembly of the handheld powered surgical device. Turning the rotation knob may act to rotate the end effector when cutting tissue or preparing to cut tissue.

The first surgical device 54752 may obtain data related to a device operating condition associated with the user operation. As described herein, the user operation may be the user 54754 pulling the trigger on the first surgical device 54752, the user 54754 pressing the firing button of the first surgical device 54752, or the user 54754 rotating the rotation knob of the first surgical device 54752. The data related to the device operating condition may include a device location, a device orientation, and/or a device directionality in relation to the surgical environment.

For example, the data related to the device operating condition may be or may include the position of the end-effector of the first surgical device 54752 in relation to the tissue meant to be cut during surgery when the user operation is performed. The data related to the device operating condition may indicate whether the end-effector is positioned proximate to the tissue or is positioned away from the when the user performs the user operation. In examples, the position of the end-effector may be determined by sensors on the end-effector that can detect its positioning relative to the tissue. In examples, the first surgical device 54572 may include a QR code near the end-effector that can detect its proximity to the tissue. In examples, the position of the end-effector may be determined by measuring the tissue impendence (e.g., which may determine whether the end-effector is cutting tissue is not cutting tissue).

For example, the data related to the device operating condition may be or may include the orientation or directionality of the end-effector of the first surgical device 54752 in relation to the tissue is it planning to cut during surgery when the user operation is performed. The orientation or direction of the end-effector may indicate whether the end-effector is cutting or preparing to cut the tissue or is oriented or is in a direction away from the tissue when the user performs the user operation. In examples, the data may be generated based on the imaging feed from the imaging system. By analyzing the live images or video of the surgical site, the position, orientation, and/or directionality of the end-effector in relation to the issue may be determined.

In examples of the first surgical device 54572 being a handheld surgical stapler, the data related to the device operating condition may be whether or not the actuation (e.g., used to perform stapling jobs) is locked. If the actuator is locked, the surgical stapler may not be proximate to the tissue or may not be allowed to perform the stapling job. If the actuator is unlocked, the surgical stapler may be proximate to the tissue or may be allowed to perform the stapling job.

The data related to the device operating condition may include the natural frequency of the ultrasonic blade or the power level of the bipolar RF. This data may be used to determine one or more of a collagen transition temperature, a blade temperature, a tissue composition, an initial tissue contact/thickness, a tissue distribution within jaws, the force/power relationship, or a tissue component conversion temperature.

The data related to the device operating condition may include a measured tissue property threshold such as tissue impendence or clamp force. The data may be a non-therapeutic monitoring sub-signal with the RF signal to identify critical structures (e.g., nerves). The data may be related to tissue classification. The data may be generator data, clamp force, device information, feedback, manufacturing data, etc./eprom, jaw force, generator reading, or generator information provided back to the hub. The data may be related to blade breakage. The data may be monitored aspects of the first surgical device 54572, the tissue, or the electronic signal itself. This data may be used as a control means for altering a control unit algorithm of the first surgical device 54572 to improve functional outcomes.

The first surgical device 54572 may determine, based on the data related to the device operating condition, whether the user operation is associated with a second surgical device separate from the first surgical device 54572. Examples of the second surgical device may include the advanced imaging device 54756, the smoke evacuator 54758, the irrigation system 54760, the cooling mechanism (not shown), the insufflation pump (not shown), or the generator(s) (not shown), etc.

The first surgical device 54572 may determine that the user operation is associated with the second surgical device based on the data related to a device operating condition indicating the user operation is invalid for the first surgical device 54572. For example, the user operation may be invalid for the first surgical device 54572 if the user is interacting with two controls that inherently are always prevented from being used together. For example, the device operating condition may require the end-effector of the first surgical device 54572 to be touching the tissue or be proximate to the tissue (e.g., meant to be cut for a surgery) for the user operation to be valid for the first surgical device.

In examples, the first surgical device 54572 may determine that the user operation is invalid for the first surgical device and may determine the user operation is associated with the second surgical device based on the location of the end-effector of the first surgical device 54572 being away from the tissue when performing the user operation (e.g., pulling the trigger of the first surgical device 54572, pressing a firing button of the first surgical device 54572, rotating a knob of the first surgical device 54572, etc.).

In examples, if the first surgical device 54572 determines that the user operation is invalid for cutting tissue with the end-effector, the first surgical device may determine the user operation is valid for controlling the advanced imaging device 54756. For example, if the end-effector is away from the tissue or the end-effector is closed, performing the operation (e.g., pulling the trigger of the first surgical device 54572, pressing a firing button) may direct a surgical scope of the advanced imaging device 54756 to capture a different area or view of the surgical site, reposition a focal center of an image of the advanced imaging device 54756, and/or to change the imaging of the advanced imaging device 54756 among fluorescing imaging, multi-spectral imaging, and visible light imaging. For example, if the end-effector is away from the tissue or the end-effector is closed, squeezing the trigger once or pressing the firing button once may configure to zoom in the advanced imaging device 54756 and squeezing the trigger twice or pressing the firing button twice may configure to zoom out the advanced imaging device 54756. For example, if the end-effector is away from the tissue or the end-effector is closed, rotating the rotating knob of the first surgical device 54756 may configure to control the advanced imaging device 54756, such as changing different areas of the advanced imaging device 54756 to view or zooming in/zooming out the advanced imaging device 54756 a certain amount.

In examples, if the first surgical device 54572 determines that the user operation is invalid for cutting tissue with the end-effector, the first surgical device 54572 may determine the user operation is valid for controlling the smoke evacuator 54758. For example, if the end-effector is away from the tissue or the end-effector is closed, performing the user operation (e.g., pulling the trigger of the first surgical device, pressing a firing button) may act to control the smoke evacuator 54758. For example, if the end-effector is away from the tissue or the end-effector is closed, squeezing the trigger once or pressing the firing button once may activate the smoke evacuator 54758 and squeezing the trigger twice or pressing the firing button twice may deactivate the smoke evacuator 54758. For example, if the end-effector is away from the tissue or the end-effector is closed, rotating the rotation knob may control how much smoke, fluid, and/or particulates that evacuate from the smoke evacuator 54758 (e.g., how much smoke is applied).

In examples, if the first surgical device 54572 determines that the user operation is invalid for cutting tissue with the end-effector, the first surgical device 54572 may determine the user operation is for controlling the irrigation system 54760. For example, if the end-effector is away from the tissue or the end-effector is closed, performing the user operation (e.g., pulling the trigger of the first surgical device, pressing a firing button) may act to control the irrigation system 54760. For example, if the end-effector is away from the tissue or the end-effector is closed, squeezing the trigger once or pressing the firing button once may activate the irrigation system 54760 and squeezing the trigger twice or pressing the firing button twice may deactivate the irrigation system 54760. For example, if the end-effector is away from the tissue or the end-effector is closed, rotating the rotation knob may control how much fluid is directed along the irrigation channel of the irrigation system 54760.

In examples, if the first surgical device 54572 determines the user operation is invalid for cutting tissue with the end-effector, the first surgical device 54572 may determine the user operation is directed to controlling another surgical device such as generator(s) (not shown), an insufflation pump (not shown), a cooling mechanism (not shown) and/or then like. For example, if the end-effector is away from the tissue or the end-effector is closed, performing the user operation (e.g., pulling the trigger of the first surgical device, pressing a firing button) may act to control the generator(s), insufflation pump, and/or cooling mechanism. For example, if the end-effector is away from the tissue or the end-effector is closed, squeezing the trigger once or pressing the firing button once may activate the generator(s), insufflation pump, or cooling mechanism and squeezing the trigger twice or pressing the firing button twice may deactivate the generator(s), insufflation pump, or cooling mechanism. For example, if the end-effector is away from the tissue or the end-effector is closed, rotating the rotation knob may control the amount of power being outputted by the generator(s), how much fluid is directed by the insufflation pump, and/or how long to use and/or the temperature of the cooling mechanism.

Based on the determination that the user operation is associated with the second surgical device, the first surgical device 54572 may generate a control signal for controlling the second surgical device.

The first surgical device 54572 may control the second surgical device via a communication interface. The communication interface may be a virtual interface separate from the first surgical device 54572. The virtual interface may show the surgical field using a surgical camera and a surgical display.

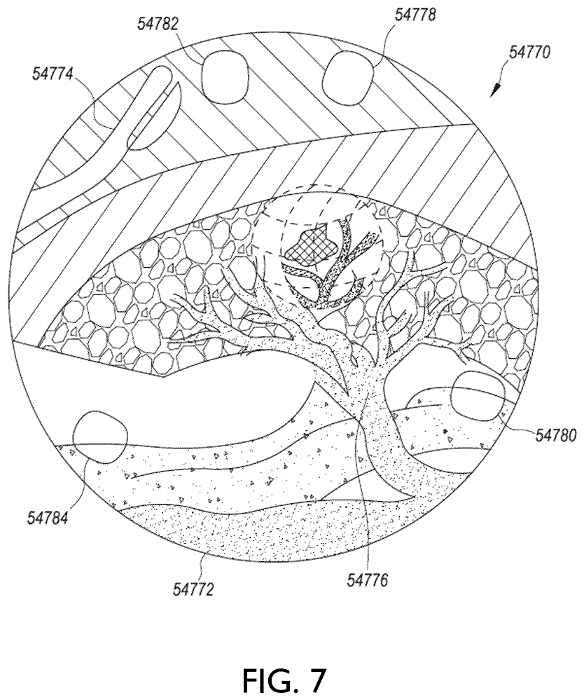

FIG. 7 illustrates a surgical field of view 54770 including a communication interface 54772 providing the ability for the first surgical device to control the second surgical device.

In examples, the virtual interface may show the position of the first surgical device 54572 (e.g., the position of the end-effector) in relation to the tissue (e.g., as shown in FIG. 7). If the end-effector is proximate to the tissue and/or is open as shown on the virtual interface, a user performing the user operation (e.g., pulling the trigger of the first surgical device, pressing a firing button) may act to control the end-effector to cut the tissue. If the end effector is not proximate to the tissue and/or is closed as shown on the virtual interface, a user performing the user operation (e.g., pulling the trigger of the first surgical device, pressing a firing button) may to control the second surgical device.

In examples, after the user operation is performed, the virtual interface may display a number of devices. The first surgical device 54572 may control different surgical devices and provide different options of how to control them (e.g., as shown in FIG. 7). In examples, the first surgical device may be used as a mouse via the virtual interface to select the surgical device it wants to control and how to control it. For example, there may be an option to control the advanced imaging system to get a different view of the surgical site. For example, there may be an option to control the generator(s) and how to set different power levels of the generator(s). For example, there may be an option to control the smoke evacuator (e.g., how much smoke to use, the length of time to use it, etc.)

As shown in FIG. 7, the surgical field of view 54770 may show data related to the device operating condition associated with the user performing the user operation. For example, the surgical field of view 54770 may be configured to display the position of the end-effector 54774 of the first surgical device in relation to the tissue 54776.

If the end-effector 54774 is proximate to the tissue 54776 and/or is open as shown on the surgical field of view 54770 when performing a user operation, the first surgical device may act to control the end-effector 54774 to cut the tissue 54776. If the end-effector 54774 is not proximate to the tissue 54776 and/or is closed as shown on the surgical field of view 54770 when performing a user operation, the first surgical device may determine that the user operation corresponds to an invalid input for operating the first surgical device and may act to control a second surgical device. After the user operation is performed and the first surgical device determines to control the second surgical device, the communication interface 54772 of the surgical field of 54770 may display a number of second surgical devices the first surgical device may control and different options of how to control them. The first surgical device may use a device position of the first surgical device within the surgical field of view 54770 to select the second surgical device to control via a user operation.

For example, the first surgical device may be used as a mouse via the communication interface 54772 to select the second surgical device the first surgical device wants to control and/or how to control it (e.g., by clicking on the second surgical device the first surgical device wants to control). The surgical field of view 54770 may include small circles displayed on different areas of the surgical field of view 54770. The circles (e.g., each circle) may represent a second surgical device to control or different controls related to the second surgical device. For example, a small circle 54778 on the upper right corner to may control the smoke evacuator, a small circle 54780 on the lower right corner may control the advanced imaging device, a small circle 54782 on the upper left corner may control the generator(s), a small circle 54784 on the lower left corner may control the irrigation system.

If the smoke evacuator is selected via the circle 54778, there may an option to control the smoke evacuator (e.g., how much smoke to use, the length of time to use it, etc.). If the advanced imaging device is selected via the circle 54780, there may be option to control the advanced imaging device to display a different view of the surgical site (e.g., reposition the focal center of the image, shift between fluorescing, multi-spectral imaging, and visible light imaging, etc.). If the generator(s) are selected via the circle 54782, there may be an option to control the generator(s) and how to set different power levels of the generator(s).

In examples, movement of the first surgical device within a visible occurrence may indicate which second surgical device the first surgical device wants to control (e.g., moving the first surgical device in a smoke plume to control the smoke evacuator). For example, if a smoke plume is obscuring a portion of the screen, the user may move the first surgical device into that area, close the jaws, and perform the user operation which may instruct the smoke evacuator to change the extraction rate or the activation state. For example, immersion of the end-effector in an occluding pool of fluid may indicate the first surgical device to control a suction of the insufflation or irrigation pump.

In examples, a surgical computing system (e.g., surgical hub) may be configured to (e.g., rather than the first surgical device) control the second surgical device. The surgical computing system may detect a user operation at the first surgical device. Data may be obtained associated with the user operation. Based on the data related to the device operating condition, the surgical computing system may determine whether the user operation is associated with the first surgical device or the second surgical device. Based on the determination that the user operation is associated with the second surgical device, a control signal may be generated for controlling the second surgical device.

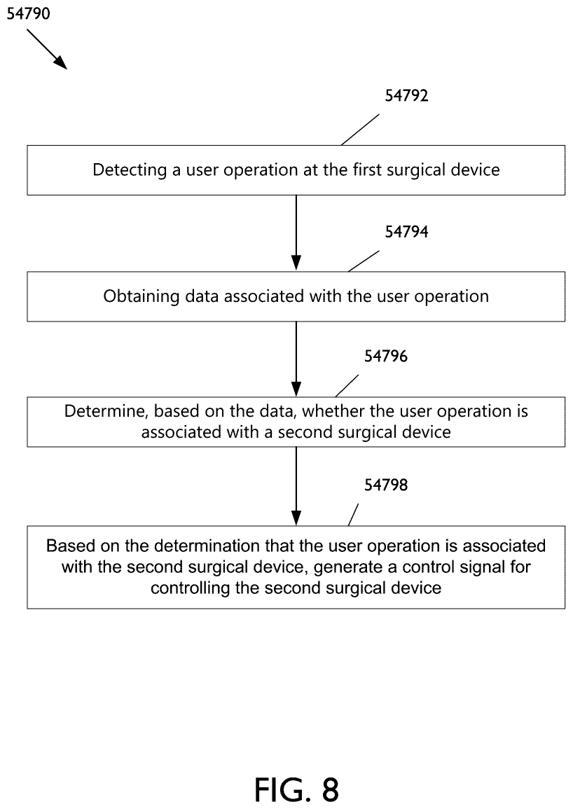

FIG. 8 illustrates a flow diagram of a process 54790 for a first surgical device controlling the operation of a second surgical device. At block 54792, a user operation may be detected at the first surgical device. The user operation may be a user pulling the trigger, the user pressing the firing button, or the user rotating the rotation knob. The first surgical device may be a handheld surgical instrument having an end-effector that can cut tissue.

At block 54794, data associated with the user operation may be obtained. The data associated with the device operating condition may be related to a device location, a device orientation, or a device directionality relative to a surgical environment within the surgical network.

At block 54796, based on the data related to the device operating condition, the first surgical device may determine whether the user operation is associated with a second surgical device separate from the first surgical device. In examples, the first surgical device may determine that the data related to the device operating condition indicates that the user operation corresponds to an invalid input for the first surgical device. Based on the determination that the data related to the device operating condition indicates that the user operation corresponds to an invalid input for operating the first surgical device, the first surgical device may determine that the user operation is associated with operating the second surgical device separate from the first surgical device.

The second surgical device may be an advanced imaging device, a smoke evacuator, an irrigation pump, generator(s), a cooling mechanism, an insufflation pump, etc. In examples, based on the determination that the data related to the device operating condition indicates that the user operation corresponds to an invalid input for operating the first surgical device, the user operation may be used to reposition a focal center of an image of the advanced imaging device or may be used to change an imaging of the advanced imaging device between fluorescing imaging, multi-spectral imaging, and visible light imaging. In examples, based on the determination that the data related to the device operating condition indicates that the user operation corresponds to an invalid input for operating the first surgical device, the user operation may be used to operate the smoke evacuator, the irrigation pump, the generator(s), the cooling mechanism, the insufflation pump, etc.

In block 54798, based on the determination that the user operation is associated with the second surgical device, the first surgical device may generate a control signal for controlling the second surgical device. The control signal for controlling the second surgical device may be sent via a communications interface. In examples, the first surgical device may communicate with the second surgical device via a surgical field of view having a communication interface providing the ability for the first surgical device to control the second surgical device.

What is claimed:

1. A first surgical device, comprising:
a processor configured to:
detect a user operation at the first surgical device;
obtain data related to a device operating condition associated with the user operation;
determine, based on the data related to the device operating condition, whether the user operation is associated with the first surgical device or a second surgical device separate from the first surgical device; and
based on the determination that the user operation is associated with the second surgical device, generate a control signal for controlling the second surgical device.

2. The first surgical device of claim 1, wherein the data related to the device operating condition is related to a device location, a device orientation, or a device directionality relative to a surgical environment.

3. The first surgical device of claim 1, wherein the processor is further configured to:
determine that the data related to the device operating condition indicates that the user operation corresponds to an invalid input for the first surgical device,
based on the determination that the data related to the device operating condition indicates that the user operation corresponds to an invalid input for operating the first surgical device, determine that the user operation is associated with operating the second surgical device separate from the first surgical device.

4. The first surgical device of claim 1, wherein the first surgical device is a handheld surgical instrument comprising an end effector, the second surgical device is an advanced imaging device, and the processor is further configured to:
based on the data related to the device operating condition indicating that the handheld surgical instrument is operating with the end effector outside a proximity of a tissue when the user operation is performed, determine that the user operation corresponds to an invalid input for operating the first surgical device;
based on the determination that the data related to the device operating condition indicates that the user operation corresponds to an invalid input for operating the first surgical device, determine that the user operation is used to reposition a focal center of an image of the advanced imaging device; and
based on the determination that the user operation is used to reposition a focal center of an image of the advanced imaging device, generate the control signal to reposition the focal center of the image of the advanced imaging device.

5. The first surgical device of claim 1, wherein the first surgical device is a handheld surgical instrument comprising an end effector, the second surgical device is an advanced imaging device, and the processor is further configured to:
based on the data related to the device operating condition indicating that the handheld surgical instrument is operating with the end effector outside a proximity of a tissue when the user operation is performed, determine that the user operation corresponds to an invalid input for operating the first surgical device;
based on the determination that the data related to the device operating condition indicates that the user operation corresponds to an invalid input for operating the first surgical device, determine that the user operation is used to change an imaging of the advanced imaging device between fluorescing imaging, multi-spectral imaging, and visible light imaging; and
based on the determination that the user operation is used to change the imaging of the advanced imaging device, generate the control signal to change the imaging of the advanced imaging device between the fluorescing imaging, the multi-spectral imaging, and the visible light imaging.

6. The first surgical device of claim 1, wherein the first surgical device is a handheld surgical instrument comprising an end-effector, the second surgical device is a smoke evacuator, and the processor is further configured to:
based on the data related to the device operating condition indicating that the handheld surgical instrument is operating with the end effector closed when the user operation is performed, determine that the user operation corresponds to an invalid input for operating the first surgical device; and
based on the determination that the data related to the device operating condition indicates that the user operation corresponds to an invalid input for operating the first surgical device, determine that the user operation is associated with operating the smoke evacuator.

7. The first surgical device of claim 1, wherein the first surgical device is a handheld surgical instrument comprising an end-effector, the second surgical device is an irrigation pump, and the processor is further configured to:
based on the data related to the device operating condition indicating that the handheld surgical instrument is operating with the end effector outside a proximity of a tissue when the user operation is performed, determine that the user operation corresponds to an invalid input for operating the first surgical device; and
based on the determination that the data related to the device operating condition indicates that the user operation corresponds to an invalid input for operating the first surgical device, determine that the user operation is associated with operating the irrigation pump.

8. The first surgical device of claim 1, wherein the data related to the device operating condition associated with the user operation is shown via a surgical field of view that is configured to display the second surgical device to control, and the processor is further configured to:
based on the surgical field of view showing that the surgical device is operating with an end effector outside a proximity of a tissue when the user operation is performed, determine that the user operation corresponds to an invalid input for operating the first surgical device; and
based on the determination that the user operation corresponds to an invalid input for operating the first surgical device, use a device position of the first surgical device within the surgical field of view to select the second surgical device to control via the user operation.

9. A method performed by a first surgical device, comprising:
detecting a user operation at the first surgical device;
obtaining data related to a device operating condition associated with the user operation;
determining, based on the data related to the device operating condition, whether the user operation is associated with the first surgical device or a second surgical device separate from the first surgical device; and
based on the determination that the user operation is associated with the second surgical device, generating a control signal for controlling the second surgical device.

10. The method of claim 9, wherein the data related to the device operating condition is related to a device location, a device orientation, or a device directionality relative to a surgical environment.

11. The method of claim 9, further comprising:
determining that the data related to the device operating condition indicates that the user operation corresponds to an invalid input for the first surgical device,
based on the determination that the data related to the device operating condition indicates that the user operation corresponds to an invalid input for operating the first surgical device, determining that the user operation is associated with operating the second surgical device separate from the first surgical device.

12. The method of claim 9, wherein the first surgical device is a handheld surgical instrument comprising an end effector and the second surgical device is an advanced imaging device, further comprising:
based on the data related to the device operating condition indicating that the handheld surgical instrument is operating with the end effector outside a proximity of a tissue when the user operation is performed, determining that the user operation corresponds to an invalid input for operating the first surgical device;
based on the determination that the data related to the device operating condition indicates that the user operation corresponds to an invalid input for operating the first surgical device, determining that the user operation is used to reposition a focal center of an image of the advanced imaging device; and
based on the determination that the user operation is used to reposition a focal center of an image of the advanced imaging device, generating the control signal to reposition the focal center of the image of the advanced imaging device.

13. The method of claim 9, wherein the first surgical device is a handheld surgical instrument comprising an end effector and the second surgical device is an advanced imaging device, further comprising:
based on the data related to the device operating condition indicating that the handheld surgical instrument is operating with the end effector outside a proximity of a tissue when the user operation is performed, determining that the user operation corresponds to an invalid input for operating the first surgical device;
based on the determination that the data related to the device operating condition indicates that the user operation corresponds to an invalid input for operating the first surgical device, determining that the user operation is used to change an imaging of the advanced imaging device between fluorescing imaging, multi-spectral imaging, and visible light imaging; and
based on the determination that the user operation is used to change the imaging of the advanced imaging device, generating the control signal to change the imaging of the advanced imaging device between the fluorescing imaging, the multi-spectral imaging, and the visible light imaging.

14. The method of claim 9, wherein the first surgical device is a handheld surgical instrument comprising an end-effector and the second surgical device is a smoke evacuator, further comprising:
based on the data related to the device operating condition indicating that the handheld surgical instrument is operating with the end effector closed when the user operation is performed, determining that the user operation corresponds to an invalid input for operating the first surgical device; and
based on the determination that the data related to the device operating condition indicates that the user operation corresponds to an invalid input for operating the first surgical device, determining that the user operation is associated with operating the smoke evacuator.

15. The method of claim 9, wherein the first surgical device is a handheld surgical instrument comprising an end-effector and the second surgical device is an irrigation pump, further comprising:
based on the data related to the device operating condition indicating that the handheld surgical instrument is operating with the end effector outside a proximity of a tissue when the user operation is performed, determining that the user operation corresponds to an invalid input for operating the first surgical device; and
based on the determination that the data related to the device operating condition indicates that the user operation corresponds to an invalid input for operating the first surgical device, determining that the user operation is associated with operating the irrigation pump.

16. The method of claim 9, wherein the data related to the device operating condition associated with the user operation is shown via a surgical field of view that is configured to display the second surgical device to control, further comprising:
based on the surgical field of view showing that the surgical device is operating with an end effector outside a proximity of a tissue when the user operation is performed, determining that the user operation corresponds to an invalid input for operating the first surgical device; and
based on the determination that the user operation corresponds to an invalid input for operating the first surgical device, using a device position of the first surgical device within the surgical field of view to select the second surgical device to control via the user operation.

17. A surgical computing system, comprising:
a processor configured to:
detect a user operation at the first surgical device;
obtain data associated with the user operation;
determine, based on the data related to a device operating condition, whether the user operation is associated with the first surgical device or the second surgical device; and
based on the determination that the user operation is associated with the second surgical device, generate a control signal for controlling the second surgical device.

18. The surgical computing system of claim 17, wherein the data related to the device operating condition is related to a device location, a device orientation, or a device directionality relative to a surgical environment.

19. The surgical computing system of claim 17, wherein the processor is further configured to:
determine that the data related to the device operating condition indicates that the user operation corresponds to an invalid input for the first surgical device,
based on the determination that the data related to the device operating condition indicates that the user operation corresponds to an invalid input for operating the first surgical device, determine that the user operation is associated with operating the second surgical device separate from the first surgical device.

20. The surgical computing system of claim 17, wherein:
the first surgical device is a handheld surgical instrument comprising an end-effector, the second surgical device is an advanced imaging device, and the processor is further configured to:
based on the data related to the device operating condition indicating that the handheld surgical instrument is operating with the end effector outside a proximity of a tissue when the user operation is performed, determine that the user operation corresponds to an invalid input for operating the first surgical device;
based on the determination that the data related to the device operating condition indicates that the user operation corresponds to an invalid input for operating the first surgical device, determine that the user operation is used to change an imaging of the advanced imaging device between fluorescing imaging, multi-spectral imaging, and visible light imaging; and
based on the determination that the user operation is used to change the imaging of the advanced imaging device, generate the control signal to change the imaging of the advanced imaging device between the fluorescing imaging, the multi-spectral imaging, and the visible light imaging.
